(19) United States
(12) Patent Application Publication
(10) Pub. No.: US 2022/0347066 A1
(43) Pub. Date: Nov. 3, 2022

(54) WATER-IN-OIL EMULSION COMPOSITION FOR ENHANCED DELIVERY OF WATER SOLUBLE SKIN BENEFIT AGENTS

(71) Applicant: Conopco, Inc., d/b/a UNILEVER, Englewood Cliffs, NJ (US)

(72) Inventors: Sivakumar ANANTHASUBRAMANIAN, Mumbai (IN); Naresh Dhirajlal GHATLIA, Southbury, CT (US); Swapnil Ravikant HEGISHTE, Mumbai (IN); Mruthyunjaya Swamy MATHAPATHI, Bangalore (IN); Amitabha MAJUMDAR, Bangalore (IN); Divya PARUCHURI, Hyderabad (IN)

(73) Assignee: Conopco, Inc., d/b/a UNILEVER, Englewood Cliffs, NJ (US)

(21) Appl. No.: 17/626,859

(22) PCT Filed: Jul. 8, 2020
(86) PCT No.: PCT/EP2020/069227

§ 371 Date: Jan. 13, 2022

(30) Foreign Application Priority Data
Jul. 23, 2019 (EP) 19187809.9

(51) Int. Cl. A61K 8/06; A61K 8/92; A61K 8/31; C11D 17/00; A61Q 19/10

(52) U.S. Cl. CPC A61K 8/064; A61K 8/92; A61K 8/315; C11D 17/006; A61Q 19/10

(57) ABSTRACT

This invention relates to a water in oil emulsion composition which provides enhanced delivery of water soluble skin benefit agent. The invention more particularly relates to a wash off composition e.g that for washing hair, body, hand or face which generally contain anionic surfactants and yet provide enhanced delivery of such agents. It does this by delivering the actives through a specific water-in-oil emulsion. The water-in-oil emulsion comprises a hydrophobic phase preferably petrolatum with preferred emulsifiers being non-ionic surfactant of specified HLB range.

### FIELD OF THE INVENTION

This invention relates to an emulsion composition which provides enhanced delivery of water soluble skin benefit agents. The invention more particularly relates to a personal care composition, more particularly a cleansing composition e.g that for washing hair, body, hand or face which generally contain anionic surfactants and yet provide enhanced delivery of water soluble actives especially cationic actives. It does this by delivering the actives through a specific water-in-oil emulsion.

### BACKGROUND OF THE INVENTION

Products that are used to clean topical surfaces of the human body are delivered through cleansing compositions. They may be used to clean the hair, face, body or hands. Most of these compositions comprise anionic surfactants either made from natural sources like soaps or of synthetic sources. Such compositions are also used to deliver benefits other than cleansing like moisturisation, skin lightening, anti aging, anti inflammatory, conditioning or antimicrobial benefits. Many antimicrobial actives have also been included in such cleansing composition like trichlorocarbanilide, triclosan, chloroxylenol, benzalkonium chloride, etc. Actives having cationic charges are considered to be highly effective as an antimicrobial active. Due to the charge neutralisation of such actives in an anionic based surfactant system, it has been a challenge to include cationic actives in such products. It is also a challenge to ensure deposition of water soluble actives in sufficient amounts through such compositions as they generally tend to get washed off with the rinse water. Compounds like polymeric cationic actives which have high charge density are even more difficult to incorporate in such systems.

Delivery of small molecular weight cationic species for topical application have been disclosed before. US2006051385 (3M) discloses antimicrobial compositions, especially those useful when applied topically, particularly to mucosal tissues (i.e., mucous membranes), including a cationic antiseptic such as biguanides and bisbiguanides such as chlorhexidine and its various salts including but not limited to the digluconate, diacetate, dimethosulfate, and diactate salts; polymeric quaternary ammonium compounds such as polyhexamethylenebiguanide; silver and various silver complexes; small molecule quaternary ammonium compounds such as benzalkoium chloride and alkyl substituted derivatives; di-long chain alkyl (C8-C18) quaternary ammonium compounds; cetylpyridinium halides and their derivatives; benzethonium chloride and its alkyl substituted derivatives; and octenidine. This patent publication generally relates to leave-on compositions and achieves the desired result by including an enhancer component. It does not teach how to ensure deposition of water soluble skin benefit actives in cleansing compositions comprising anionic surfactants.

Therefore, there exists a need in the art to provide for enhanced delivery of waters soluble or cationic actives in cleansing compositions comprising high amount of anionic surfactants. The present inventors have achieved this by including the actives in a water-in-oil emulsion also including a hydrophobic component like petrolatum or wax and an emulsifier, preferably a non-ionic surfactant having a specific HLB range. The present inventors have also surprisingly found that the present invention is more effective in solid cleansing composition and the stability is not as good when formulated in liquid compositions.

It is thus an object of the present invention to provide for a composition comprising anionic surfactants for enhanced delivery of water soluble skin benefit agents.

It is another object of the present invention to provide for enhanced delivery of cationic antimicrobial actives through such wash off compositions.

It is yet another object of the present invention to provide for enhanced delivery of such actives through a soap composition.

### SUMMARY OF THE INVENTION

According to the first aspect of the present invention there is provided a water-in-oil emulsion composition for enhanced delivery of water soluble skin benefit agent having a solubility in water at 25° C. of at least 0.001 wt %, comprising:

(a) an aqueous phase comprising at least one said water soluble skin benefit agent;

(b) a hydrophobic phase chosen from petrolatum or wax; and

(c) an emulsifier.

A preferred aspect relates to the emulsion composition wherein the emulsifier is a non-ionic surfactant.

A second aspect of the present invention relates to a process to prepare the water-in-oil emulsion composition of the first aspect comprising the steps of:
(a) Mixing the emulsifier with the hydrophobic phase at a temperature in the range of 20-95° C.;
(b) mixing a solution/dispersion of the skin benefit agent in water maintained at a temperature of 20 to 95° C., to the mixture of step (a).

A third aspect of the present invention relates to a cleansing composition comprising the water-in-oil emulsion composition of the first aspect and a cosmetically acceptable base comprising an anionic surfactant.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilized in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. In other words, in specifying any ranges of values, any particular upper value can be associated with any particular lower value.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method or process relating to the invention) mutatis mutandis.

By a water-in-oil emulsion composition is meant a composition which is in the emulsion form where the continuous phase is an oil and the dispersed phase is water which may include water soluble actives.

By ‘a cleansing composition’ as used herein, is meant to include a composition for topical application to skin, hair and/or scalp of mammals, especially humans which is generally applied to the topical surface to provide cleansing benefits. Such a composition is generally applied on to the desired topical surface of the body for a period of time from a few seconds to up to several minutes. When the period of time of application is low say of the order of a few seconds to a few minutes after which the composition is rinsed off with water or wiped away, such a composition is known as a cleansing composition or a wash-off composition. When the composition is applied for longer period of time say from several minutes to up to 24 hours and washed off usually during the process of normal personal cleansing, such a composition is known as a leave-on composition. The composition as per the present invention is preferably of the wash-off type. It includes any product applied to a human body for also improving appearance, cleansing, odor control or general aesthetics. The cleansing composition of the present invention is preferably in the form of a gel, semisolid or a solid composition especially preferred being a bar composition for delivering the product as a shampoo, conditioner, handwash, facewash or bodywash product. It is more preferably used for disinfecting the hand or other parts of the human body.

The present invention relates to a water-in-oil emulsion composition for enhanced delivery of water soluble skin benefit agent. The emulsion is a water in oil composition. Thus the emulsion composition is one where the oil (or hydrophobic) phase is the continuous phase in which the water phase is dispersed in the form of droplets. The water soluble skin benefit agent is included in the water (or aqueous phase). It is ensured that the water-in-oil emulsion is maintained in a stable state through the use of an emulsifier.

Any water soluble skin benefit agent which satisfies the water solubility criterion of 0.001 wt %, preferably 0.002 wt %, further more preferably 0.005 wt % at 25° C. may be used. It is preferably one or more of an antimicrobial polymeric cationic active; water soluble vitamins and derivatives thereof; a prebiotic for skin health microbiome chosen from glycerol, sugar, glucose, fructose; a water soluble sunscreen; alpha or beta hydroxy acids, probiotic; peptides; water soluble hyaluronic acid, collagen and their salts; water soluble natural extracts; water soluble antimicrobials like itaconic acid; self tanning compounds like dihydroxy acetone; caffeine; water soluble insect repellents like IR3535; and water soluble moisturizers including glycerol quat or hydroxyethyl urea.

Of the above actives the preferred ones are one or more of an antimicrobial polymeric cationic active; water soluble vitamins selected from one or more of vitamin C, B3, B5 or N-methylnicotinamide and derivatives thereof; caffeine; water soluble insect repellents including IR3535; peptides; water soluble antimicrobials including itaconic acid; a water soluble sunscreen; a prebiotic for skin health microbiome chosen from glycerol, sugar, glucose, fructose; water soluble moisturizers including glycerol quat or hydroxyethyl urea; alpha or beta hydroxy acids; or water soluble natural extracts.

More preferred skin benefit agent is selected from an antimicrobial polymeric cationic active; water soluble vitamins selected from one or more of vitamin C, B3, B5 or N-methylnicotinamide; a water soluble sunscreen; alpha or beta hydroxy acids; peptides; water soluble hyaluronic acid, collagen and their salts; self tanning compounds like dihydroxy acetone; caffeine; water soluble insect repellents like IR3535.

Further preferred skin benefit agent is selected from one or more of antimicrobial polymeric cationic active; a water soluble sunscreen; alpha or beta hydroxy acids; water soluble vitamins selected from one or more of vitamin C,B3, B5 or N-methylnicotinamide; caffeine; water soluble insect repellents including IR3535; and peptides.

Yet further preferred actives are antimicrobial polymeric cationic active; water soluble vitamins selected from one or more of vitamin C,B3, B5 or N-methylnicotinamide; a water soluble sunscreen; alpha or beta hydroxy acids; water soluble insect repellents including IR3535; and peptides.

The most preferred active is a polymeric cationic molecule.

Polymeric cationic molecule which may be preferably included in the emulsion composition is one or more of poly diallyl dimethyl ammonium chloride (PDADMAC) or chitosan, preferably PDADMAC.

PDADMAC is homopolymer of diallyldimethyl ammonium chloride (DADMAC. The preferred PDADMAC molecular weight for use in this invention is in the range of 2,00,000-20,00,000, preferably 4,00,000-6,00,000. It is a high charge density cationic polymer of around 126 and with a viscosity in the range of 10,000-20,000 mPas. The polymer is available under the trade name of Merquat-100 (INCI: Polyquaternium-6) from Lubrizol Inc.

Water soluble vitamin or derivatives thereof which may be included are chosen from one or more of vitamin C, E, B3, B5 or N-methylnicotinamide, more preferably Vitamin B3. Water soluble sunscreen which may be included is chosen from one or more of 2-phenylbenzimidazole-5-sulfonic acid (sold as Enulizole) or ethylhexyl salicylate (sold as Neoheliopan OS). Alpha hydroxy acid which may be included is chosen from one or more of lactic acid, glycolic acid or salicylic acid preferably lactic acid. Probiotics which may be included in the composition are preferably bacterial lysates. Peptides when included in the composition are preferably hydrolysed amino acids with 10 or less peptide units. Water soluble insect repellent is preferably IR3535 which has the chemical name ethyl butyl acetyl aminopropionate.

The water soluble skin benefit agent is preferably included in the water-in-oil emulsion composition at 5 to 50%, more preferably 10 to 40%, by weight of the emulsion composition.

The hydrophobic phase is chosen from petrolatum or wax, preferably petrolatum. The hydrophobic phase may also comprise smaller amounts of other hydrophobic materials like fatty acids, triglycerides, or silicones.

Petrolatum which is known as petroleum jelly is a purified mixture of semi-solid hydrocarbons obtained from petroleum with a carbon chain length of 25 or higher. The petroleum jelly has excellent moisturizing property and has a melting point ranging from a little below to a few degrees above 37° C. It is colorless or pale yellow (when not highly distilled), translucent and devoid of taste and smell when pure. It is insoluble in water. Petrolatum is also generally known as petroleum jelly. Preferred petrolatum for use in the present invention is one having a slip melting point in the range of 45 to 75° C. The hydrophobic phase is preferably included in 20 to 60%, more preferably 20 to 50% by weight of the water-in-oil emulsion composition.

“Wax” as used herein refers to a class of organic compounds that characteristically comprise long alkyl chains. Typically the waxes are plastic (malleable) at about 25° C. “Wax ester” as used herein means ester which is comprised by a wax. The wax preferably has a melting point from 40° C. to 200° C., more preferably from 50° C. to 120° C.

The wax for use in the present invention is preferably suitable for use in a cosmetic composition. The wax may be natural wax and/or synthetic wax. Such waxes are often selected from hydrocarbon waxes and ester waxes but the wax preferably comprises wax ester. In some preferred embodiments, the wax comprises beeswax, rice bran wax, montan wax, spermaceti wax, carnauba wax, candelilla wax, sugarcane wax, insect wax, petroleum jelly, or a mixture thereof. More preferably, the wax comprises beeswax, rice bran wax, montan wax, carnauba wax, petroleum jelly or a mixture thereof. Even more preferably, the wax is beeswax.

The emulsifier for use in emulsifying the water in the oil and keeping it in a stable condition is preferably a non-ionic surfactant. Preferred surfactants for use in the emulsion composition of the invention have an HLB value of less than 10, preferably between 2 and 7.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various non-ionic surfactants are given below:
A value <10: Lipid soluble (water insoluble)
A value >10: Water soluble
A value from 4 to 8 indicates an anti-foaming agent
A value from 7 to 11 indicates a W/O (water in oil) emulsifier
A value from 12 to 16 indicates oil in water emulsion
A value from 11 to 14 indicates a wetting agent
A value from 12 to 15 is typical of detergents
A value of 16 to 20 indicates a solubiliser or hydrotrope.

Suitable emulsifiers for use in the water-in-oil emulsion compositions of the invention are selected from sorbitan monostearate, sorbitan monooleate or combinations thereof. Sorbitan monostearate is sold under the brand name SPAN-60 and sorbitan monooleate is sold under the brand name SPAN-80. It is preferred that the emulsion is prepared using both of these emulsifiers.

The emulsifier is included in 2 to 10%, preferably 2 to 8% by weight of the emulsion composition.

Water or the aqueous phase forms the dispersed phase in the water-in-oil emulsion. It is preferably present in 40 to 60% preferably 40 to 55% by weight of the emulsion.

The emulsion is preferably in the liquid state at ambient temperature (30° C.). The viscosity of the emulsion is preferably in the range of 50,000 to 2,00,000 cps, more preferably in the range of 1,00,000 to 1,50,000 cps as measured using a Brookfield viscometer with LV4 spindle at 6 rpm.

The water-in-oil emulsifier is preferably prepared using the following process to ensure maximal inclusion of the water soluble skin benefit agent in the water/aqueous phase.

The steps comprise (a) mixing the emulsifier with the hydrophobic phase at a temperature in the range of 20-95° C.; followed by (b) mixing a solution/dispersion of the water soluble skin benefit agent in water maintained at a temperature of 20 to 95° C., to the mixture of step (a). The preferred temperatures for both step (a) and step (b) are in the range of 70 to 85° C.

The invention also relates to a cleansing composition comprising the water-in-oil emulsion composition of the first aspect and a cosmetically acceptable base comprising an anionic surfactant. The cosmetically acceptable base comprises water in addition to the anionic surfactant. A particularly preferred anionic surfactant is soap. The emulsion composition is preferably included in 1 to 20%, preferably 2 to 10% by weight of the cleansing composition.

The soap is preferably C8-C24 soap, more preferably 010-C20 soap and most preferably C12-018 soap. The cation of the soap can be alkali metal, alkaline earth metal or ammonium. Preferably, the cation of the soap is selected from sodium, potassium or ammonium. More preferably the cation of the soap is sodium or potassium.

A typical fatty acid blend consists of 5 to 30% coconut fatty acids and 70 to 95% tallow fatty acids by weight of soap. Fatty acids derived from other suitable oils/fats such as groundnut, soybean, tallow, palm, palm kernel, etc. may also be used in other desired proportions.

The cosmetically acceptable base comprising the anionic surfactant forms the rest of the composition other than the water-in-oil emulsion composition of the first aspect of the invention. Thus, the cosmetically acceptable base generally forms 80 to 99% by weight of the cleansing composition.

When present, the anionic surfactant e.g. soap, is preferably present in an amount of 1 to 90%, preferably from 10 to 85%, more preferably 25 to 75% by weight of the cleansing composition. The cleansing composition is preferably in the form of a solid or semi solid form, most preferably in a solid form. Preferred solid compositions are in the shape of a soap bar.

Other anionic surfactants are preferably selected from alkyl ether sulphate, primary alkyl sulphate, secondary alkyl sulphonates, alkyl benzene sulphonates, or ethoxylated alkyl sulphates. The anionic surfactant other than soap which is preferred in the cleansing composition of the invention is an alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C6 and C16.

Preferred compositions may include other known ingredients such as perfumes, pigments, preservatives, emollients, sunscreens, gelling agents and thickening agents. Choice of these ingredients will largely depend on the format of the composition.

Water is a preferred carrier. When water is present, it is preferably present in at least 1%, more preferably at least 2%, further more preferably at least 5% by weight of the composition. When water is the carrier, a preferred cleansing composition comprises 10 to 40%, more preferably 12 to 25% by weight water.

The invention also relates to a method of delivering skin benefit agents to skin comprising the step of applying the composition of the invention on to the desired skin surface. This is followed by substantially removing the composition from the surface after a specified amount of time. Usually people spend about 10 seconds to 2 minutes washing their body parts and it is expected that in this time frame the desired deposition of the actives should have taken place. The time for washing is preferably from 10 second to one minute more preferably from 10 seconds to 30 seconds. The removal of the composition from the surface may be achieved by wiping the composition off the surface using a suitable wipe. Alternately it may be achieved by rising the surface with water to be substantially free of the composition. The composition is usually applied to the desired skin surface after diluting the composition with water. The composition may be diluted with water to a concentration of 1 to 50%, preferably 2 to 20% by weight of the diluted solution.

The invention will now be illustrated with the help of the following non-limiting examples.

### EXAMPLES

Effect of Inclusion of a Polymeric Cationic Antimicrobial Active in a Water-In-Oil Emulsion

The following water-in-oil emulsion (as in Table—1 below) was prepared and incorporated in a soap bar.

TABLE 1

| Ingredient | Wt % |
|---|---|
| Petrolatum | 32 |
| Sorbitan Monooleate (SMO) | 2 |
| Sorbitan Monostearate (SMS) | 1.5 |
| PDADMAC (Merquat 100) | 25 |
| Water | To 100 |

Emulsion making process:
1. Dose the pre-weighed quantity of petrolatum in mixing vessel and heat the mass till 82° C.
2. Put this mass under continuous stirring and add the emulsifiers (SMO and SMS) till the temperature is stabilized to 82° C.
3. Measure required quantity of Merquat-100 and DM (demineralised) water in another vessel and heat the mass till 82° C. under continuous stirring. It is useful to have dispersed and continuous phase at similar temperatures above 80° C. to avoid cooling of emulsion mass when mixed.
4. Put the petrolatum with emulsifier mass (Oil phase) under Silverson homogenizer and set the Silverson at 2000 RPM. Dose the Merquat-100 with water (Water phase) in the mixing vessel at the rate of 70 ml/min and gradually increase the Silverson speed to 5000 RPM.
5. Continue the mixing for 20 minutes
6. After emulsification, cool the mass under ambient setting till 30° C.
7. Check the viscosity (@ 30° C.) with LV4 spindle, 6 rpm. It is preferably higher than 125000 cps.
8. Transfer the material in storage jar for further use.

The above emulsion composition was used to prepare a soap bar (Table—2) by the conventional milled and plodded route.

TABLE 2

| | |
|---|---|
| Anhydrous Soap | 70 |
| Glycerin | 2 |
| Talc | 4 |
| PDADMAC Emulsion* | 4 |
| Fragrance | 1 |
| Sodium Chloride | 0.8 |
| Water and minors | Up to 100 |

*PDADAMAC Emulsion in the above table refers to the emulsion as prepared in Table-1

Antimicrobial activity was measured as follows:
1. Prewash step (7 days): A non-antimicrobial soap bar was given to each volunteer to use for bathing, washing hands, washing forearms etc.
2. Volunteers were instructed to refrain from using leave on product (sun screen, hand sanitizer, skin moisturizers, lotion, cream, oil and antimicrobial products) till completion of the study.
3. Volunteers were asked to come to the study site after seven days of prewash.
4. On day 8, one forearm was washed with placebo soap bar and other forearm washed with test soap bar as mentioned below.
   (a) Temperature of tap water was maintained at 24° C.±2° C.
   (b) The soap was wetted/dipped for 10 secs in water.
   (c) Each forearm was wetted with water (100 ml±10 ml).
   (d) The soap was rubbed 10 times back and forth across the length of the forearm.
   (e) Water was applied (3 ml±1 ml) on forearm and lathered for 30 seconds.
   (f) The lather was retained on the forearm for 20 seconds
   (g) The forearm was washed with water for 1 minute to make sure that the soap was washed off completely
   (h) The excess water was removed by patting dry using sterile tissue paper
5. Post 30 min wash, 10 μl of E. coli (10536) from 10⁸ stock was applied on defined circular area on forearm skin (7 cm² circle) for 5 mins. (10⁸ culture stock was prepared in 10 mM sodium phosphate buffer using 18 to 20 hrs old broth culture. The optical density OD was adjusted to 0.8 at 620 nm to attain 10⁸ counts)
6. After 5 mins contact time of E. coli on forearm skin, it was recovered by cup scrub method (ASTM method, E2752-10) using 1.5 ml of extraction buffer.
7. Then each sample was serially diluted in 9 ml of D/E (Dey Engley neutralizing broth) and respective dilutions were plated on MacConkey agar media.
8. Plates were incubated at 37° C. for 24 h to grow E. coli and then colonies were counted and the log cfu/ml was calculated by using standard microbiology method.

The residual E. Coli after 30 minutes of wash using the two bars are given in the tables 3 and 4 below

TABLE 3

| | | Residual E.Coli |
|---|---|---|
| Example | Composition | (log cfu/ml) |
| A | Control soap bar | 3.0 |
| B | Soap bar with petroleum jelly emulsion | 2.6 |

TABLE 4

| | | Residual E.Coli |
|---|---|---|
| Example | Composition | (log cfu/ml) |
| C | Control soap bar | 3.6 |
| 1 | Soap bar with 1% PDADMAC in petroleum | 2.8 |
| | jelly emulsion as per the invention | |

The data in the tables above indicate that the soap bar with 1% PDADMAC in a water in oil emulsion as per the invention (Example 1) gives a 0.8 log reduction in E. Coli (with respect to the control Example C) which is better than the log reduction of 0.4 achieved with a similar bar prepared without PDADMAC (Example B) with respect to its control bar (Example A).

### Example D-E: Delivery of PDADMAC without Using the Water-In-Oil Emulsion of the Invention

A non antibacterial soap bar was prepared (Example D) whose composition is given below. Another soap bar with 1% of PDADMAC in it was prepared by direct addition in the soap without use of the water-in-oil emulsion of the present invention. The antimicrobial efficacy of these two soap compositions (Table—5) were measured using the same protocol as given earlier and the data is summarised in Table—6 below:

TABLE 5

| | Control Soap | Soap bar with 1% |
|---|---|---|
| | bar (Exp D) | PDADMAC (exp E) |
| Anhydrous Soap | 68 | 67 |
| Glycerin | 6 | 4 |
| Talc | 6 | 6 |
| PDADMAC | — | 1 |
| Petrolatum | — | 1.6 |
| Fragrance | 1 | 1 |
| Sodium Chloride | 0.8 | 0.8 |
| Water and minors | To 100 | To 100 |

TABLE 6

| | | Residual E. coli |
|---|---|---|
| Example | Composition | (log cfu/ml) |
| D | Control soap bar | 3.8 |
| E | Soap bar with 1% PDADMAC | 3.7 |

The data in Table—6 indicates that there is no improvement in antimicrobial efficacy (Example E) when PDADMAC is present in a soap bar (when not included through the emulsion of the present invention) as compared to a control soap without PDADMAC (Example D).

1. A water-in-oil emulsion composition for enhanced delivery of water-soluble skin benefit agent having a solubility in water at 25° C. of at least 0.001 wt %, comprising:
(a) an aqueous phase comprising at least one said water soluble skin benefit agent;
(b) a hydrophobic phase chosen from petrolatum or wax; and
(c) an emulsifier;
wherein the water-soluble skin benefit agent is selected from an antimicrobial polymeric cationic active,
wherein the skin benefit agent is included in 5 to 50% by weight of the emulsion composition,
wherein the hydrophobic phase is included in 10 to 40% by weight of the emulsion composition; and
wherein the emulsifier is included in 2 to 10% by weight of the emulsion composition.

2. (canceled)

3. The water-in-oil composition as claimed in claim 1, wherein the petrolatum has a slip melting point in the range of 45 to 75° C.

4. The water-in-oil composition as claimed in claim 1, wherein the emulsifier is a non-ionic surfactant.

5. The water-in-oil composition as claimed in claim 3, wherein the non-ionic surfactant has an HLB value of less than 10.

6. The water-in-oil composition as claimed in claim 4, wherein the emulsifier is selected from sorbitan monostearate, sorbitan monooleate or combinations thereof.

7. (canceled)

8. The water-in-oil composition as claimed in claim 1, wherein the antimicrobial polymeric cationic active is polydiallyl dimethyl ammonium chloride or chitosan.

9. (canceled)

10. (canceled)

11. (canceled)

12. A process to prepare the water-in-oil emulsion composition as claimed in claim 1, comprising the steps of:
(a) mixing the emulsifier with the hydrophobic phase at a temperature in the range of 20 to 95° C.; and
(b) mixing a solution/dispersion of the skin benefit agent in water maintained at a temperature of 20 to 95° C., to the mixture of step (a).

13. A cleansing composition comprising 1 to 20% by weight of the water-in-oil emulsion composition as claimed in claim 1 and a cosmetically acceptable base comprising an anionic surfactant.

14. The cleansing composition as claimed in claim 13, wherein the anionic surfactant comprises soap and the cleansing composition is a soap bar.

15. The water-in-oil emulsion as claimed in claim 5, where the HLB value is between 2 and 7.
